(19) United States
(12) Patent Application Publication
(10) Pub. No.: US 2023/0338119 A1
(43) Pub. Date: Oct. 26, 2023

(54) ORTHODONTIC SYSTEM AND DESIGN METHOD AND PREPARING METHOD THEREOF

(71) Applicant: SHANGHAI SMARTEE DENTI-TECHNOLOGY CO., LTD., Shanghai (CN)

(72) Inventors: XINGXING WANG, SHANGHAI (CN); GANG WU, SHANGHAI (CN); JUNFENG YAO, SHANGHAI (CN)

(21) Appl. No.: 18/344,904

(22) Filed: Jun. 30, 2023

Related U.S. Application Data
(63) Continuation of application No. PCT/CN2021/118853, filed on Sep. 16, 2021.

(30) Foreign Application Priority Data
Dec. 31, 2020 (CN) 202011638977.8
Dec. 31, 2020 (CN) 202023325100.1

(51) Int. Cl. A61C 7/08; A61C 7/00

(52) U.S. Cl. CPC A61C 7/08; A61C 7/002

(57) ABSTRACT

The orthodontic system includes shell-shaped dental appliances, where a dental appliance body is further provided with an eruption portion spaced apart from one or more teeth not grown to preset eruption parameters. As an orthodontic plan proceeds, the eruption portion on each of the shell-like dental appliances has a constant or basically constant column structure. The shell-like dental appliances have an effect of correcting irregularities of teeth. The eruption portion is configured to accommodate teeth not grown to the preset eruption parameters. A gap between the eruption portion and erupting teeth makes the dental appliance body reserve a space for tooth growth above the erupting tooth, such that the shell-like dental appliances cannot interfere with natural growth of teeth when being worn. In addition, the eruption portion with the constant or basically constant column structure is simpler to design and use and can serve as a standard attachment.

### CROSS REFERENCE TO RELATED APPLICATIONS

The present disclosure is a continuation of PCT Patent Application No. PCT/CN2021/118853, entitled “ORTHODONTIC TREATMENT SYSTEM, AND DESIGN METHOD AND PREPARATION METHOD THEREOF,” filed Sep. 16, 2021, which claims priority to Chinese patent application No. 202023325100.1, entitled “ORTHODONTIC TREATMENT SYSTEM, AND DESIGN METHOD AND PREPARATION METHOD THEREOF,” filed Dec. 31, 2020, and Chinese patent application No. 202011638977.8, entitled “ORTHODONTIC TREATMENT SYSTEM, AND DESIGN METHOD AND PREPARATION METHOD THEREOF,” filed Dec. 31, 2020, each of which is incorporated by reference herein in its entirety.

### TECHNICAL FIELD

The present disclosure relates to the field of medical devices and in particular, to the field of invisible orthodontics, and more particularly to an orthodontic system, a design method thereof, and a method for preparing an orthodontic system, applied to the orthodontic treatment of adolescents during a period of tooth replacement.

### BACKGROUND

Invisible aligners are recognized by many consumers for their aesthetics, wearing comfort and good orthodontic effect. Among them, adolescents are a special part of the cases, for whom there is a certain stage in which deciduous teeth fall out and permanent teeth erupt, which is different from the adult permanent teeth orthodontic treatment. In a process of orthodontic treatment with the invisible aligner, in response to the teeth being treated according to the same structure as the actual model in the mouth, there is a possibility that a part of the invisible aligner corresponding to teeth that have not erupted is covered by the invisible aligner. As the teeth erupt, a corresponding position of the invisible aligner may not have enough space for eruption of the teeth due to covering adjacent gums, which may affect normal eruption of the teeth or make the invisible aligner unable to be worn after the teeth erupt.

Some methods in the prior art adopt a follow-up design for eruption space and tooth eruption, but there are certain problems with the above methods. For example, there are many comprehensive factors in a process of tooth eruption in the patient's mouth. If the eruption space is not designed reasonably in design of an orthodontic plan, there is a risk that the eruption space and the teeth may come into contact with each other and generate forces that affect the normal eruption of the teeth. In addition, if the prediction for tooth eruption speed and eruption parameters is not accurate during the follow-up design process, there may be errors in the eruption design, resulting in the patient being unable to wear the appliance normally.

All of these effects are undesirable during the orthodontic treatment process. Therefore, it is of great significance to develop an orthodontic system that has a simple eruption space design and does not affect normal growth and eruption of the teeth as the orthodontic treatment plan proceeds, as well as a design method thereof.

### SUMMARY

The present disclosure mainly aims to provide an orthodontic system, a design method thereof, and a method for preparing an orthodontic system, which can be applied to orthodontic treatment of adolescents during a period of tooth replacement to allow simultaneous tooth eruption during correction of dental malformations by a shell-like dental appliance.

An orthodontic system is provided according to some embodiments of the present disclosure, the orthodontic system includes at least one shell-like dental appliance configured to gradually adjust each of teeth other than unerupted teeth from an initial position to a target orthodontic position according to an orthodontic plan while allowing the teeth to erupt naturally. Each of the at least one shell-like dental appliance includes a dental appliance body including a geometric structure for accommodating multiple maxillary teeth or multiple mandibular teeth, and the dental appliance body is further provided with at least one eruption portion for accommodating one or more teeth not grown to preset eruption parameters; as the orthodontic plan proceeds, the eruption portion on each of the at least one shell-like dental appliances has a constant or substantially constant column structure, and there is a gap between an inner surface of each of the at least one eruption portion and an outer surface of each of the one or more teeth not grown to preset eruption parameters; where the preset eruption parameters include parameters of a tooth or teeth formed after full eruption of one or more ungrown or incompletely grown teeth.

In some embodiments, the preset eruption parameters include a size, a position, a shape and an orientation of the tooth or teeth formed after the full eruption of the one or more ungrown or incompletely grown teeth.

In some embodiments, the constant or substantially constant column structure is arranged based on a size, a position, a shape and an orientation of a tooth or teeth formed after full eruption of the one or more teeth not grown to the preset eruption parameters.

Specifically, as the orthodontic plan proceeds, adjacent teeth of the one or more teeth not grown to the preset eruption parameters is subjected to orthodontic movement, and thus there is an appropriate adjustment for the column structure of the eruption portion for a smooth transition to allow a smooth connection between the eruption portion and other parts of the shell-like body. In some embodiments, the column structure has a size 1.02 to 1.05 times of a size of the tooth or teeth formed after the full eruption of the one or more teeth not grown to the preset eruption parameters; the column structure is oriented at an angle of 0 to 5 degrees to a long axis of the tooth or teeth formed after the full eruption of the one or more teeth not grown to the preset eruption parameters; the column structure is positioned at an offset of 0 to 1 mm from a coordinate value in a spatial three-dimensional coordinate system of each of vertices that constitute a position of the tooth or teeth formed after the full eruption of the one or more teeth not grown to the preset eruption parameters; the column structure is shaped at an offset of 0 to 1 mm from a coordinate value in the spatial three-dimensional coordinate system of each of vertices that constitute a shape of the tooth or teeth formed after the full eruption of the one or more teeth not grown to the preset eruption parameters. The constant or substantially constant column structure as described in this embodiment is achieved in the above range of size, position, shape, and orientation.

In some embodiments, the constant or substantially constant column structure is further arranged based on a size, a position, a shape and an orientation of a tooth or teeth on an opposite jaw corresponding to the one or more teeth not grown to the preset eruption parameters, so that the constant or substantially constant column structure does not interfere with a maxillomandibular occlusal relationship.

In some embodiments, the constant or substantially constant column structure is arranged based on both first preset parameters of mesial adjacent teeth and second preset parameters of distal adjacent teeth of the one or more teeth not grown to the preset eruption parameters.

In some specific embodiments, the first preset parameters include the following parameters of the mesial adjacent teeth: a maximum dimension in a buccolingual diameter direction, a maximum dimension in a mesial-distal direction, and a maximum dimension of a height of a tooth in a long axis direction; and the second preset parameters include the following parameters of the distal adjacent teeth: a maximum dimension in the buccolingual diameter direction, a maximum dimension in the mesial-distal direction, and a maximum dimension of the height of the tooth in the long axis direction.

In some embodiments, the constant or substantially constant column structure includes a labial/buccal surface, a lingual surface and an occlusal surface, where the labial/buccal surface is a flat surface or a curved surface with a smooth transition to a labial/buccal surface of the mesial adjacent teeth and the distal adjacent teeth, the lingual surface is a flat surface or a curved surface with a smooth transition to a lingual surface of the mesial adjacent teeth and the distal adjacent teeth, and the occlusal surface is a flat surface or a curved surface with a smooth transition to an occlusal surface of the mesial adjacent teeth and the distal adjacent teeth.

In some embodiments, the constant or substantially constant column structure is further arranged based on a size, a position, a shape and an orientation of teeth on an opposite jaw corresponding to the one or more teeth not grown to the preset eruption parameters, so that the constant or substantially constant column structure does not interfere with the maxillomandibular occlusal relationship.

In some embodiments, the constant or substantially constant column structure is arranged based on third preset parameters of distal adjacent teeth and mesial adjacent teeth of the one or more teeth not grown to the preset eruption parameters.

In some specific embodiments, the third preset parameters include: a maximum dimension in a buccolingual diameter direction, a maximum dimension in a medial-distal direction of the distal adjacent teeth, and a maximum dimension of a height of a tooth in a long axis direction of the mesial adjacent teeth.

In some embodiments, the constant or substantially constant column structure includes a labial/buccal surface, a lingual surface and an occlusal surface, where the labial/buccal surface is a flat surface or a curved surface with a smooth transition to a labial/buccal surface of the mesial adjacent teeth and the distal adjacent teeth, the lingual surface is a flat surface or a curved surface with a smooth transition to a lingual surface of the mesial adjacent teeth and the distal adjacent teeth, and the occlusal surface is a flat surface or a curved surface with a smooth transition to an occlusal surface of the mesial adjacent teeth and the distal adjacent teeth.

In some embodiments, the constant or substantially constant column structure is further arranged based on a size, a position, a shape, and an orientation of teeth on an opposite jaw corresponding to the one or more teeth not grown to the preset eruption parameters, so that the constant or substantially constant column structure does not interfere with the maxillomandibular occlusal relationship.

In some embodiments, the constant or substantially constant column structure is a cylindrical structure, an elliptical cylindrical structure, or a multi-prismatic column structure with not less than four lateral ribs.

In some embodiments, a geometric structure on the shell-like dental appliance is configured to gradually adjust each of the teeth other than the unerupted teeth from the initial position to the target orthodontic position.

A design method of an orthodontic system is further provided according to some embodiments of the present disclosure, including following operations S1 to S4.

S1. obtaining a digital dental model: obtaining the digital dental model, where the digital dental model includes a digital tooth model and a digital gingival model;

S2. segmenting and identifying the digital dental model: segmenting the digital dental model into a separate digital gingival model and multiple digital crown models in a one-to-one correspondence to the teeth; identifying and marking data indicating teeth which do not erupt or do not fully erupt;

S3. virtually designing an orthodontic plan: designing the multiple digital tooth crown model in a one-to-one correspondence to the teeth virtually so that each of the multiple digital tooth crown models in a one-to-one correspondence to the teeth gradually changes from an initial position to a target orthodontic position to obtain a series of intermediate digital dental models;

S4. designing the orthodontic system:

designing at least one shell-like dental appliance configured to gradually adjust each of teeth other than unerupted teeth from an initial position to a target orthodontic position according to an orthodontic plan while allowing the teeth to erupt naturally. Each of the at least one shell-like dental appliance includes a dental appliance body including a geometric structure for accommodating multiple maxillary teeth or multiple mandibular teeth, and the dental appliance body is further provided with at least one eruption portion for accommodating one or more teeth not grown to preset eruption parameters;

as the orthodontic plan proceeds, the eruption portion on each of the at least one shell-like dental appliances has a constant or substantially constant column structure, and there is a gap between an inner surface of each of the at least one eruption portion and an outer surface of each of the one or more teeth not grown to preset eruption parameters; where the preset eruption parameters include parameters of a tooth or teeth formed after full eruption of one or more ungrown or incompletely grown teeth.

In one embodiment of the above design method, the geometric structure on the at least one shell-like dental appliance other than the eruption portion is configured to gradually adjust each of the teeth other than the unerupted teeth from the initial position to the target orthodontic position.

In addition, a method for preparing an orthodontic system is further provided according to some embodiments of the present disclosure, a shell-like dental appliance in the orthodontic system obtained according to the design method described above is prepared by using a thermocompression film molding or an additive preparing process to obtain a series of the shell-like dental appliances.

The present disclosure has the following advantages over the prior art.

The orthodontic system for invisible orthodontics provided according to the present disclosure includes a shell-like dental appliance. The shell-like dental appliance includes a dental appliance body, and the dental appliance body is further provided with an eruption portion, and the eruption portion on each of the shell-like dental appliances has a constant or substantially constant column structure as the orthodontic plan proceeds. The shell-like dental appliance has the effect of correcting dental malformations, and at the same time, the eruption portion of the dental appliance body is provided to receive the teeth not grown to the preset eruption parameters. The gap between the eruption portion and the teeth not grown to the preset eruption parameters is provided so that the dental appliance body, when worn, reserves space for the teeth to grow over the one or more teeth not grown to the preset eruption parameters. Therefore, each of the shell-like dental appliances of the entire orthodontic system does not interfere with the natural growth of the teeth when worn. In addition, the present disclosure provides the eruption portion with the constant or substantially constant column structure, which makes design and use of each dental appliance and the eruption portion simpler. The eruption portion can be used as a standard attachment, which can be inserted on the dental model by selecting the standard attachment during use.

Implementation of any aspect of the present disclosure can achieve some or all of the above advantages.

### BRIEF DESCRIPTION OF THE DRAWINGS

FIG. 1 is schematic structural view of a shell-like dental appliance in a front view according to a first embodiment of the present disclosure;

FIG. 2 is a schematic structural view of a shell-like dental appliance in a side view according to the first embodiment of the present disclosure;

FIG. 3 is a schematic structural view of another shell-like dental appliance in a front view according to the first embodiment of the present disclosure;

FIG. 4 is a schematic structural view of another shell-like dental appliance in a side view according to the first embodiment of the present disclosure;

FIG. 5 is a schematic structural view of a shell-like dental appliance according to the first embodiment of the present disclosure when being worn.

FIG. 6 is a schematic diagram of a design method of an orthodontic system according to a second embodiment of the present disclosure;

FIG. 7 is a schematic structural view of a shell-like dental appliance in a front view according to the second embodiment of the present disclosure;

FIG. 8 is a schematic structural view of a shell-like dental appliance in a side view according to the second embodiment of the present disclosure;

FIG. 9 is a schematic structural view of another shell-like dental appliance in a front view according to the second embodiment of the present disclosure;

FIG. 10 is a schematic structural view of another shell-like dental appliance in a side view according to the second embodiment of the present disclosure;

FIG. 11 is a schematic structural view of a shell-like dental appliance in a front view according to a fourth embodiment of the present disclosure;

FIG. 12 is a schematic structural view of a shell-like dental appliance in a side view according to the fourth embodiment of the present disclosure;

FIG. 13 is a schematic structural view of a shell-like dental appliance in a front view according to a fifth embodiment of the present disclosure;

FIG. 14 is a schematic structural view of a shell-like dental appliance in a side view according to the fifth embodiment of the present disclosure.

Reference numerals: shell-like dental appliance (100, 200, 400, 500); eruption portion (120, 220), dental appliance body (110, 210, 410, 510); eruption cavity (420, 520).

### DETAILED DESCRIPTION OF THE EMBODIMENTS

In the description of the present disclosure, it should be noted that “one or more teeth not grown to preset eruption parameters”, i.e., “one or more ungrown or incompletely grown teeth”, is also referred to as “erupting tooth” or “erupted tooth”.

In the description of the present disclosure, it should be noted that orientation or positional relationships indicated by the terms “center”, “up”, “down”, “left”, “right”, “vertical”, “horizontal”, “inside”, “outside”, and the like are based on orientation or positional relationships shown in the accompanying drawings, which is only intended to facilitate and simplify the description of the present disclosure, rather than indicating or implying that the device or element referred to must have a particular orientation, be constructed and operated in a particular orientation, and therefore should not be construed as a limitation on the present disclosure. In addition, the terms “first”, “second”, and “third” are used for descriptive purposes only and should not be construed as indicating or implying relative importance.

As used in this specification, the singular forms “one”, “a”, and “the” include plural objects, unless expressly stated otherwise.

The present disclosure is further described below in combination with specific embodiments.

### First Embodiment

This embodiment provides an orthodontic system including at least one shell-like dental appliance 100 configured to gradually adjust each of teeth other than unerupted teeth from an initial position to a target orthodontic position according to an orthodontic plan while allowing the teeth to erupt naturally according to an orthodontic plan. Referring to FIG. 1 to FIG. 5, which are schematic structural views of the shell-like dental appliance of this embodiment, the shell-like dental appliance includes a dental appliance body 110. The dental appliance body 110 includes a geometric structure for accommodating multiple maxillary teeth or multiple mandibular teeth. The dental appliance body 110 is further provided with at least one eruption portion 120 for accommodating one or more teeth not grown to preset eruption parameters. The eruption portion 120 on each of the shell-like dental appliances 100 has a constant or substantially constant column structure as the orthodontic plan proceeds, and a gap is set between an inner surface of the eruption 120 and an outer surface of the teeth not grown to the preset eruption parameters. The preset eruption parameters include parameters of a tooth or teeth formed after full eruption of one or more ungrown or incompletely grown teeth.

The orthodontic system for invisible orthodontics provided in this embodiment is suitable for orthodontic treatment of adolescents in a period of tooth replacement. Due to a long period of an orthodontic plan, six months or even longer, patients in the period of tooth replacement need to design a space used to accommodate erupting teeth, influence of the erupting teeth on the orthodontic plan should be considered. When designing the orthodontic system, it is ensured that the erupting teeth are not subjected to forces generated by interaction with the shell-like dental appliance, which affects the eruption. Otherwise, the shell-like body for orthodontic treatment may cover gingiva above the erupting teeth and would inhibit the growth of the erupting teeth.

Specifically, the shell-like dental appliance 100 (also noted as a dental appliance) of this embodiment has an effect of correcting tooth malocclusion while the eruption portion 120 on the dental appliance body 110 is provided to receive the teeth not grown to the preset eruption parameters. A gap between the inner surface of the eruption portion 120 and the teeth not grown to the preset eruption parameters allows the dental appliance body 110 to reserve a space for tooth growth above the one or more teeth not grown to the preset eruption parameters when worn. Therefore, the shell-like dental appliance 100 does not interfere with the natural growth of the teeth when worn. Herein, the orthodontic system of this embodiment is suitable for an orthodontic plan with multiple orthodontic stages, where the eruption portion 120 of each of the dental appliance body 110 has a constant or substantially constant column structure as the orthodontic plan proceeds, so that each of the dental appliance bodies 110 does not touch the teeth never grown to the preset eruption parameters throughout the orthodontic system. In addition, the structure of the eruption portion 120 makes design and use of the respective dental appliance and the eruption portion 120 simpler, and it (eruption portion 120) is used as a standard attachment that can be inserted on a dental model by selecting that standard attachment when in use.

In some embodiments, the preset eruption parameters include a size, a position, a shape, and an orientation of the tooth or teeth formed after the full eruption of the one or more ungrown or incompletely grown teeth. Herein, the size, position, shape, and orientation may be a size, position, shape, and orientation of the incompletely grown teeth obtained based on CBCT of a patient, or a size, position, shape, and orientation of the ungrown or incompletely grown teeth obtained based on one or more denture banks, or a size, position, shape, and orientation of the ungrown or incompletely grown teeth based on statistics under big data.

In some embodiments, the constant or substantially constant column structure is arranged based on the size, position, shape, and orientation of the tooth or teeth formed after the full eruption of the one or more teeth not grown to the preset eruption parameters.

Specifically, as the orthodontic plan proceeds, adjacent teeth of the one or more teeth not grown to the preset eruption parameters are subjected to orthodontic movement, and thus there is an appropriate adjustment of the column structure of the eruption portion 120 for a smooth transition to allow a smooth connection between the eruption portion 120 and other parts of the shell-like body.

In some embodiments, the column structure has a size 1.02 to 1.05 times of a size of the tooth or teeth formed after the full eruption of the one or more teeth not grown to the preset eruption parameters. More specifically, the size of the column structure is based on the size of the tooth after full eruption, which is a fixed size and does not change. Therefore, the column structure designed based on the fixed size and further enlarged by 1.02 to 1.05 times has a larger size than that of the tooth or teeth formed after the full eruption of the one or more teeth not grown to the preset eruption parameters, ensuring that the one or more teeth not grown to the preset eruption parameters always remain out of contact with the inner surface of the eruption portion 120 (eruption cavity) during the eruption process.

The column structure is oriented at an angle of 0 to 5 degrees to a long axis of the tooth or teeth formed after the full eruption of the one or more teeth not grown to the preset eruption parameters. More specifically, the orientation of the column structure is based on an orientation of the tooth or teeth formed after the full eruption of the one or more teeth not grown to the preset eruption parameters, which is a definite orientation. Therefore, the orientation of the column structure based on this definite orientation is designed to have a greater range of angles than the orientation of the tooth or teeth formed after the full eruption of the one or more teeth not grown to the preset eruption parameters. That is, the angular orientation is expanded by 0 to 5 degrees based on a long axis of unerupted or incompletely erupted teeth, ensuring that the unerupted or incompletely erupted teeth remain out of contact with the eruption portion 120 (eruption cavity) formed during the eruption process.

The column structure is positioned at an offset of 0 to 1 mm from a coordinate value in a spatial three-dimensional coordinate system of each of vertices that constitute a position of the tooth or teeth formed after the full eruption of the one or more teeth not grown to the preset eruption parameters. More specifically, based on the position after full eruption of the erupting tooth, which is a definite position, the position of each of the vertices of the column structure is offset outwardly from an inside of the eruption portion 120. It should be noted that the design of the orthodontic system is carried out based on a digital dental model which is composed of multiple triangular facets in a unified three-dimensional coordinate system, and each vertex in each of the triangular facets has its corresponding spatial coordinate value in the three-dimensional coordinate system. The position of the tooth or teeth formed after full eruption of the one or more teeth not grown to the preset eruption parameters is determined based on the spatial coordinate values of each vertex that constitute the position. That is, the individual vertex of the erupting tooth is used as a reference for an expansion of the 0 to 1 mm offset, which ensures that the erupting teeth remain out of contact with the eruption portion 120 (erupted cavity) formed during the eruption process.

The column structure is shaped at an offset of 0 to 1 mm from a coordinate value in the spatial three-dimensional coordinate system of each of vertices that constitute a shape of the tooth or teeth formed after the full eruption of the one or more teeth not grown to the preset eruption parameters. More specifically, the shape of the column structure is based on the shape of the tooth or teeth formed after the full eruption of the one or more teeth not grown to the preset eruption parameters, which is a definite shape, and therefore the shape of the column structure is determined based on the above-mentioned definite shape. It should be noted that the design of the orthodontic system is carried out based on the digital dental model, which is composed of multiple triangular facets in the uniform three-dimensional coordinate system, and each vertex in each of the triangular facets has its corresponding spatial coordinate value in the three-dimensional coordinate system. The shape of the tooth or teeth formed after the full eruption of the one or more teeth not grown to the preset eruption parameters is based on the spatial coordinate values of each vertex that constitute the shape, while the shape of the column structure has a larger range of offset than the shape of the tooth or teeth formed after the full eruption of the teeth not grown to the preset eruption parameters. That is, the individual vertex of the unerupted or incomplete erupting teeth is used as a reference for an expansion of 0 to 1 mm offset, which ensures that the eruption portion 120 (eruption cavity) has a smooth transition to its adjacent geometric structures and remains out of contact with the erupting tooth throughout the eruption process. The constant or substantially constant column structure as described in this embodiment is achieved with the range of size, position, shape, and orientation described above.

In some embodiments, the constant or substantially constant column structure is further arranged based on a size, a position, a shape, and an orientation of a tooth or teeth on an opposite jaw corresponding to the one or more teeth not grown to the preset eruption parameters, so that the constant or substantially constant column structure does not interfere with maxillomandibular occlusal relationship setting. Herein, the corresponding teeth on the opposite jaw refer to teeth on the opposite jaw that are occluded with the one or more teeth not grown to the preset eruption parameters, by which an occlusal surface of the constant or substantially constant column structure is designed. The occlusal surface of the constant or substantially constant column structure is designed as a flat, curved, or a structure with protrusions and recesses to be matched with opposing teeth. The occlusal surface of the eruption portion 120 is designed according to the opposing teeth, so that the eruption portion 120 is cuspid-fossa matching with the opposing teeth, or so that the occlusal surface of the eruption portion 120 is provided with protrusions and recesses to be matched with the occlusal surface of the opposing teeth.

In some embodiments, the constant or substantially constant column structure is arranged based on both first preset parameters of mesial adjacent teeth and second preset parameters of distal adjacent teeth of the one or more teeth not grown to the preset eruption parameters.

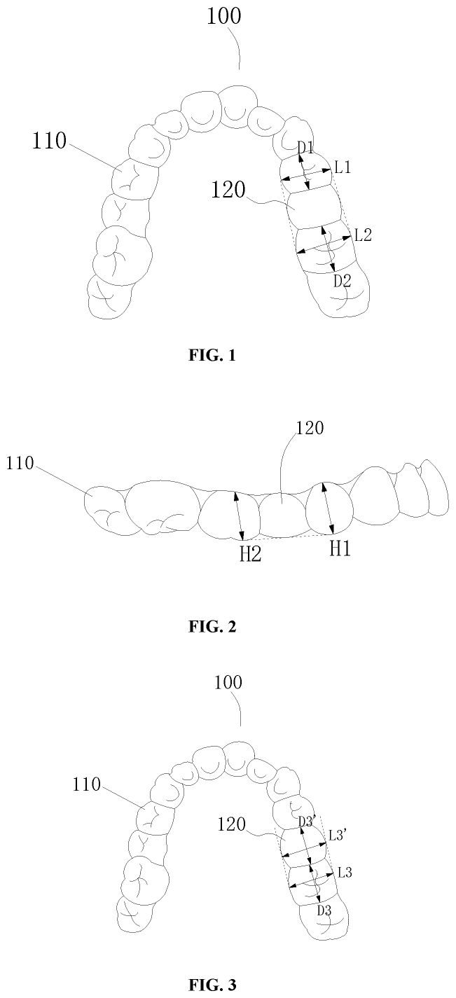

In some specific embodiments, the first preset parameters include the following parameters of the mesial adjacent teeth: a maximum dimension L₁ in a buccolingual diameter direction, a maximum dimension D₁ in a mesial-distal direction and a maximum dimension H₁ of a height of a tooth in a long axis direction; the second preset parameters include the following parameters of the distal adjacent teeth: a maximum dimension L₂ in the buccolingual diameter direction, a maximum dimension D₂ in the mesial-distal direction and a maximum dimension H₂ of the height of the tooth in the long axis direction. In one embodiment, as shown in FIG. 1 and FIG. 2, the eruption portion 120 of the shell-like dental appliance 100 is provided to encase eruption of a second premolar. At this time, the first preset parameters are the maximum dimension L₁ in the buccolingual diameter direction, the maximum dimension D₁ in the mesial-distal direction, and the maximum dimension H₁ in the height of the tooth in the long axis direction of a first premolar; the second preset parameters are the maximum dimension L₂ in the buccolingual diameter direction, the maximum dimension D₂ in the mesial-distal direction, and the maximum dimension H₁ of the height of the tooth in the long axis direction of a first molar. The determination of the constant or substantially constant column structure based on the first and second preset parameters is performed by determining the dimension of the constant or substantially constant column structure in the buccolingual diameter direction by means of arithmetic averaging, weighted averaging, etc. between L₁ and L₂; determining the dimension of the constant or substantially constant column structure in the mesial-distal direction by means of arithmetic averaging, weighted averaging, etc. between D₁ and D₂, determining the dimension of the constant or substantially constant column structure in the long axis direction of the tooth by means of arithmetic averaging, weighted averaging, etc. between H₁ and H₂. As a result, the eruption portion 120 may fit the size and shape of the one or more teeth not grown to the preset eruption parameters throughout the orthodontic plan to the maximum extent.

In some embodiments, the constant or substantially constant column structure includes a labial/buccal surface, a lingual surface, and an occlusal surface. Herein, the labial/buccal surface is a flat surface or a curved surface with a smooth transition to a labial/buccal surface of the mesial adjacent teeth and the distal adjacent teeth, the lingual surface is a flat surface or a curved surface with a smooth transition to a lingual surface of the mesial adjacent teeth and the distal adjacent teeth, and the occlusal surface is a flat surface or a curved surface with a smooth transition to an occlusal surface of the mesial adjacent teeth and the distal adjacent teeth. This setup structure allows the shell-like dental appliance 100 to have a smoother shell-like structure that wraps around the teeth and less foreign body sensation in the mouth when worn by the patient. It should be noted that the constant or substantially constant column structure means that the shape, size, position, and orientation of the eruption portion 120 are consistent. Since the teeth may move as the orthodontic treatment proceeds during the orthodontic process of wearing a series of shell-like dental appliances, the eruption portion 120 may have a partial spatial adaptation for a smooth transition connection with a geometric structure of the adjacent teeth. Alternatively, in some embodiments, the constant or substantially constant column structure may also include only the labial/buccal and lingual surfaces, but not include the occlusal surface.

In some embodiments, the constant or substantially constant column structure is further arranged based on the size, position, shape, and orientation of the teeth on the opposite jaw corresponding to the one or more teeth not grown to the preset eruption parameters so that the constant or substantially constant column structure does not interfere with the maxillomandibular occlusal relationship setting. The occlusal surface of the eruption portion 120 of this embodiment is designed as a flat, curved or a structure with protrusions and recesses to be matched with the opposing teeth. The occlusal surface of the eruption portion 120 is designed according to the opposing teeth so that the eruption portion 120 is cuspid-fossa matching with the opposing teeth, or so that the occlusal surface of the eruption portion 120 is a structure with protrusions and recesses to be matched with the occlusal surface of the opposing teeth.

In some embodiments, the constant or substantially constant column structure is arranged based on third preset parameters of distal adjacent teeth and mesial adjacent teeth of the one or more teeth not grown to the preset eruption parameters.

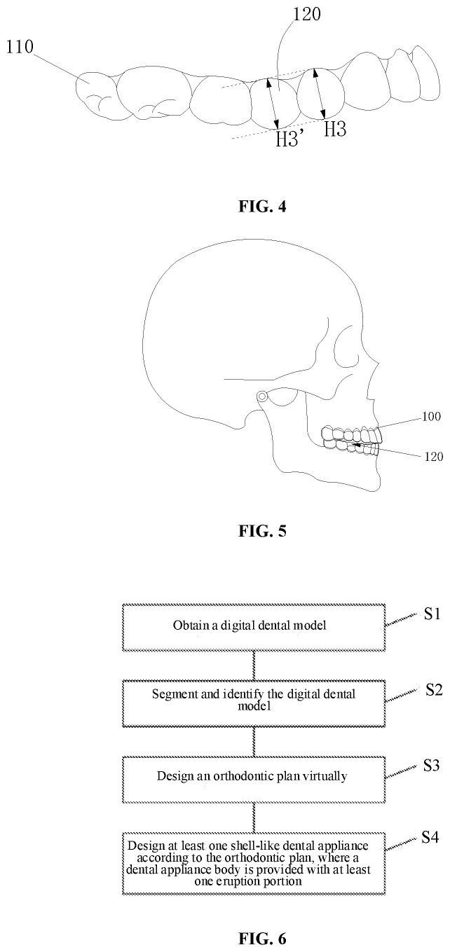

In some specific embodiments, the third preset parameters include: a maximum dimension in a buccolingual diameter direction, a maximum dimension in a mesial-distal direction of the distal adjacent teeth, and a maximum dimension of a height of a tooth in a long axis direction of the mesial adjacent teeth. In some specific embodiments, as shown in FIG. 3 and FIG. 4, the eruption portion 120 on the shell-like dental appliance is provided to encase the eruption of the second premolar. At this time, the third preset parameters are a maximum dimension L₃ in the buccolingual diameter direction, a maximum dimension D₃ in the mesial-distal direction, and a maximum dimension H₃ of the height of the tooth in the long axis direction of the first molar, which are used to determine a maximum dimension L₃′ in the buccolingual diameter direction, a maximum dimension D₃′ in the mesial-distal direction, and a maximum dimension H₃′ in the height of the long-axis direction of the mesial adjacent teeth of the eruption portion 120 of the second premolar. The column structure designed based on the maximum dimension in the buccolingual diameter direction, the maximum dimension in the medial-distal direction of the distal adjacent teeth and the maximum dimension of the height of the teeth in the long-axis direction of the medial adjacent tooth is sufficient to accommodate the one or more teeth not grown to the preset eruption parameters, and such that the dimension of the column structure is slightly larger than that of the one or more teeth not grown to the preset eruption parameters, so as to ensure that a gap can be set between the column structure and the erupting teeth.

In some embodiments, the constant or substantially constant column structure includes a labial/buccal surface, a lingual surface, and an occlusal surface. The labial/buccal surface is a flat surface or a curved surface with a smooth transition to a labial/buccal surface of the mesial adjacent teeth and the distal adjacent teeth, the lingual surface is a flat surface or a curved surface with a smooth transition to a lingual surface of the mesial adjacent teeth and the distal adjacent teeth, and the occlusal surface is a flat surface or a curved surface with a smooth transition to an occlusal surface of the mesial adjacent teeth and the distal adjacent teeth. This setup structure allows the shell-like dental appliance 100 to have a smoother shell-like structure that wraps around the teeth and less foreign body sensation in the mouth when worn by the patient.

In some embodiments, the constant or substantially constant column structure is further arranged based on the size, position, shape, and orientation of the teeth on the opposite jaw corresponding to the one or more teeth not grown to the preset eruption parameters so that the constant or substantially constant column structure does not interfere with the maxillomandibular occlusal relationship setting. The occlusal surface of the eruption portion 120 of this embodiment is designed as a flat, curved or a structure with protrusions and recesses to be matched with the opposing teeth. The occlusal surface of the eruption portion 120 is designed according to the opposing teeth so that the eruption portion 120 is cuspid-fossa matched with the opposing teeth, or so that the occlusal surface of the eruption portion 120 is a structure with protrusions and recesses to be matched with the occlusal surface of the opposing teeth.

In some embodiments, the constant or substantially constant column structure is a cylindrical structure, an elliptical cylinder, or a multi-pronged column structure with not less than four lateral ribs, which may be set according to a number and type of erupting teeth, or can be adaptively selected according to an eruptive gap existing between adjacent teeth.

In some embodiments, a geometric structure on the shell-like dental appliance 100 other than the eruption portion 120 causes teeth other than unerupted teeth to be gradually adjusted from the initial position to the target orthodontic position. That is, the eruption portion 120 in this embodiment only reserves space for growth of the one or more teeth not grown to the preset eruption parameters, so that the shell-like dental appliance 100 does not interfere with the natural growth of the erupting teeth, but the eruption portion 120 in this embodiment does not have a corrective effect on abnormally growing erupting teeth. That is, if the one or more teeth not grown to the preset eruption parameters are abnormally growing teeth, the eruption portion 120 of the present embodiment is also set according to the erupting teeth without corrective intervention. Instead, the geometric structure on the shell-like dental appliance 100 other than the eruption portion 120 is configured to gradually adjust the teeth other than the unerupted teeth from the initial position to the target orthodontic position. That is, the geometric structure other than the eruption portion 120 has a corrective effect on remaining teeth other than the erupting teeth, allowing the teeth to be aligned without interfering with the eruption of the teeth.

### Second Embodiment

This embodiment provides a design method of an orthodontic system. As shown in FIG. 6, which is a schematic diagram of the design method of this embodiment, the orthodontic system is any of the orthodontic systems as described in Embodiment 1, and the design method includes operations S1 to S4 as follows.

In operation S1, a digital dental model is obtained, and the digital dental model includes a digital tooth model and a digital gingival model.

In operation S2, the digital dental model is segmented into a separate digital gingival model and multiple digital crown models in a one-to-one correspondence to the teeth, and data indicating unerupted or incompletely erupted teeth is identified and marked.

In operation S3, the multiple digital crown models in a one-to-one correspondence to the teeth are designed virtually so that each of the multiple digital crown models in a one-to-one correspondence to the teeth gradually changes from an initial position to a target orthodontic position to obtain a series of intermediate digital dental models.

In operation S4, at least one shell-like dental appliance 200 configured to gradually adjust teeth from the initial position to the target orthodontic position while allowing the teeth to erupt is designed according to the orthodontic plan where the shell-like dental appliance 200 includes a dental appliance body 210, the dental appliance body 210 includes a geometric structure for accommodating multiple maxillary teeth or multiple mandibular teeth, the dental appliance body 210 is further provided with at least one eruption portion 220 for accommodating one or more teeth not grown to preset eruption parameters. As the orthodontic plan proceeds, the eruption portion 220 on each of the shell-like dental appliances 200 has a constant or substantially constant column structure, and a gap is set between an inner surface of the eruption portion 220 and an outer surface of the teeth not grown to the preset eruption parameters.

Specifically, in the design method described in this embodiment, a digital maxillary dental model and a mandibular dental model in step S1 is obtained by any of the following methods: a laminar X-ray scanning (CAT scanning), a digital tomography (CT) scanning, a cone beam CT scanning (CBCT), a magnetic resonance imaging (MRI), an intraoral optical scanning, etc. to obtain a digital model representing an original tooth layout. Alternatively, a plaster cast of the patient's tooth is made by conventional means, and then the plaster cast may be scanned by a scanning device such as a laser scanning device, a CT scanning device to obtain the digital model representing the original tooth layout.

Specifically, in the design method described in this embodiment, the digital dental model in step S2 is segmented in the following non-limiting embodiments.

In S200, a first type of feature points on the digital dental model to be segmented are selected, and the digital dental model is a triangular facet model.

In S201, a second type of feature points in the digital dental model are classified based on the first type of feature points and a tooth to which each of the second type of feature points belongs is determined.

In S202, the second type of feature points belonging to each tooth are merged separately to obtain a digital tooth region of each single tooth after segmentation of the digital dental model.

The above-mentioned first type of feature points are triangular facet vertices selected based on the digital dental model and used to guide segmentation of each individual tooth in a jaw, and the second type of feature points are triangular facet vertices selected based on the digital dental model and used to characterize an overall shape of the digital dental model. That is, the first type of feature points are used to guide the segmentation of the jaw, and the second type of feature points are feature points used in the specific segmentation of the jaw. Through the segmentation guidance of the first type of feature points, the second type of feature points can be accurately classified to each tooth, thereby improving segmentation accuracy of the jaw.

Segmentation for a single tooth is achieved by integrally selecting the first type of feature points on the digital dental model, and then classifying and reassembling the second type of feature points on the digital dental model based on the first type of feature points. The two types of feature points are integrally selected based on the digital dental model, and classification information of the feature points covers overall classification features of the digital dental model. Therefore, even if there is noisy data in the model, the noisy data will be evenly distributed to the global data, resulting in a high fault tolerance of the entire segmentation method. In addition, the single tooth can be segmented more accurately to ensure integrity of each tooth.

Further, in step S2, the teeth on the tooth model after cutting are identified and marked, and the specific implementation of identifying and marking the data indicating the teeth not grown to the preset eruption parameters is as follows: firstly, a tooth position is identified, and then a volume of the identified tooth is compared with a volume of a standard tooth, and when the volume of the identified tooth is smaller than the volume of the corresponding standard tooth within a certain threshold, the tooth is marked as the tooth not grown to the preset eruption parameters. The threshold is, for example, half of the volume of the standard tooth.

More specifically, the tooth position identification may use the following methods. In step 1, a first priori model, a second priori model and a third priori model are built. Herein, the first priori model includes collecting a spacing of each two adjacent teeth in the existing tooth model and the number of missing teeth corresponding to the spacing, and calculating probability distribution function values for the spacing of different numbers of missing teeth. The second priori model includes collecting feature vector representing a position of each tooth in the existing dental model, and calculating a probability distribution function value for the feature vector at least representing position of the teeth with the same number. The third prior model includes collecting tooth position arrangement of each two adjacent teeth in the existing dental model without missing teeth, or with different numbers of missing teeth, and calculating a probability distribution function value for the tooth position arrangement. In step 2, a feature vector representing the position of each tooth of the tooth model to be tested and a spacing between two adjacent teeth are acquired. In step 3, the tooth position of the tooth model to be tested is determined based on Hidden Markov Model. The identification of the tooth position is performed according to the above method, after which the tooth volume is compared with that in the standard tooth model according to the tooth position marker, such as using a change in a coordinate value of a feature point within a certain threshold, and determining whether the tooth is marked as the tooth not grown to the preset eruption parameters.

Specifically, in the design method described in this embodiment, in step S3, the single digital crown model is virtually designed so that the single digital crown model is gradually changed from an initial position to a target orthodontic position to obtain a series of intermediate digital dental models. Herein, the initial position is an original layout of the teeth before an orthodontic treatment begins, or any stage of the orthodontic treatment process, and the target orthodontic position is any stage after the orthodontic treatment begins, which is a later stage or later stages of the original layout of the teeth. The target orthodontic position is a position of the final orthodontic effect obtained by the physician and medical designer according to the patient's request and intraoral situation, or the target orthodontic position is recommended according to an intraoral digital design software, based on similar cases, or more targeted adjustments is made to patient treatment according to recommendation results.

Specifically, in the design method described in this embodiment, in step S4, the orthodontic system is designed, in which at least one shell-like dental appliance 200 that gradually adjusts the teeth from the initial position to the target orthodontic position while allowing the teeth to erupt according to the orthodontic plan is designed. The shell-like dental appliance 200 is used in any orthodontic phase of the orthodontic plan, such as an initial phase or a final phase of the orthodontic treatment. The eruption portion 220 having the constant or substantially constant column structure is provided as a standard attachment for easy use by clinicians or other users who may directly select this standard attachment for insertion into the dental model when designing the shell-like dental appliance 200.

In some embodiments, the constant or substantially constant column structure is arranged based on a size, a position, a shape, and an orientation of the tooth or teeth formed after full eruption of the one or more teeth not grown to the preset eruption parameters.

Specifically, as the orthodontic plan proceeds, adjacent teeth of the one or more teeth not grown to the preset eruption parameters is subjected to orthodontic movement, and thus there is an appropriate adjustment of the column structure of the eruption portion 220 for a smooth transition to allow a smooth connection between the eruption portion 220 and other parts of the shell-like body. In some embodiments, a size of the column structure is 1.02 to 1.05 times of a size of the tooth or teeth formed after the full eruption of the one or more teeth not grown to the preset eruption parameters. More specifically, the size of the column structure is based on the size of the tooth or teeth formed after the full eruption of the one or more teeth not grown to the preset eruption parameters, which is a fixed size and does not change. Therefore, the column structure designed based on the fixed size mentioned above has a larger size than that of the tooth or teeth formed after the full eruption of the tooth or teeth not grown to the preset parameters, which ensures that the unerupted or incompletely erupted teeth remain out of contact with an eruption cavity formed during an eruption process.

The column structure is oriented at an angle of 0 to 5 degrees to an orientation of a long axis of the tooth or teeth formed after the full eruption of the one or more teeth not grown to the preset eruption parameters. More specifically, the orientation of the column structure is based on the orientation of the tooth or teeth formed after the full eruption of the one or more teeth not grown to the preset eruption parameters, which is a definite orientation. Therefore, the orientation of the column structure determined based on the definite orientation has a larger range of orientation angles compared with the orientation of the tooth or teeth formed after the full eruption of the teeth not grown to the preset eruption parameters. That is, the orientation angle is expanded by 0 to 5 degrees based on the long axis of the teeth not grown to the preset eruption parameters, ensuring that the unerupted or incompletely erupted teeth remain out of contact with the eruption portion 220 (eruption cavity) formed during the eruption process.

The column structure is positioned at an offset of 0 to 1 mm from a coordinate value in a spatial three-dimensional coordinate system of each of vertexes that constitute a position of the tooth or teeth formed after the full eruption of the one or more teeth not grown to the preset eruption parameters. More specifically, based on the position of the tooth or teeth formed after the full eruption of the one or more teeth not grown to the preset eruption parameters, which is a definite position, the position of each vertex of the column structure is offset outwardly from an inside of the eruption portion 120 based on the definite position. It should be noted that the design of the orthodontic system is carried out based on a digital dental model, which is composed of multiple triangular facets in a unified three-dimensional coordinate system, and each vertex in each of the triangular facets has its corresponding spatial coordinate value in the three-dimensional coordinate system. The position of the tooth or teeth formed after the full eruption of the one or more teeth not grown to the preset eruption parameters is determined based on the spatial coordinate values of each of the vertices that constitutes the position. That is, each vertex of the erupted tooth is used as a reference for the expansion of the 0 to 1 mm offset, which ensures that the erupting teeth remain out of contact with the eruption portion 120 (eruption cavity) formed during the eruption process.

The column structure is shaped at an offset of 0 to 1 mm from a coordinate value in the spatial three-dimensional coordinate system of each of vertexes that constitute a shape of the tooth or teeth formed after the full eruption of the one or more teeth not grown to the preset eruption parameters. More specifically, the shape of the column structure is based on the shape of the teeth formed after the full eruption of the teeth not grown to the preset eruption parameters, which is a definite shape. Therefore, the shape of the column structure is determined based on the above definite shape. It should be noted that the design of the orthodontic system is based on the digital dental model, which is composed of multiple triangular facets in the unified three-dimensional coordinate system, and each vertex in each of the triangular facets has its corresponding spatial coordinate value in the three-dimensional coordinate system. The shape of the tooth or teeth formed after the full eruption of the one or more teeth not grown to the preset eruption parameters is based on the spatial coordinate value of each of the vertices that constitute the shape. The shape of the column structure has a greater range of offset than the shape of the teeth formed after the full eruption of the teeth not grown to the preset eruption parameters. That is, each vertex of the unerupted or incompletely erupted teeth is used as a reference for the expansion of the 0 to 1 mm offset, so that the eruption portion 120 (eruption cavity) is smoothly transitioned to its adjacent geometric structures and remains out of contact with the erupting teeth during the eruption process. The constant or substantially constant column structure as described in this embodiment is achieved in the range of the size, position, shape, and orientation as described above.

In some embodiments, the constant or substantially constant column structure is further arranged based on a size, a position, a shape, and an orientation of teeth on an opposite jaw corresponding to the one or more teeth not grown to the preset eruption parameters, so that the constant or substantially constant column structure does not interfere with the maxillomandibular occlusal relationship setting. Herein, the corresponding teeth on the opposite jaw refer to teeth on the opposite jaw that are occluded with the one or more teeth not grown to the preset eruption parameters. The occlusal surface of the constant or substantially constant column structure is designed based on the corresponding teeth on the opposite jaw. The occlusal surface of the constant or substantially constant column structure is designed as a flat, curved, or a structure with protrusions and recesses to be matched with the opposing teeth. The occlusal surface of the eruption portion 220 is designed based on the opposing teeth so that the eruption portion 220 is cuspid-fossa matching with the opposing teeth, or so that the occlusal surface of the eruption portion 220 is a structure with protrusions and recesses to be matched with the occlusal surface of the opposing teeth.

In some embodiments, the constant or substantially constant column structure is arranged based on both first preset parameters of mesial adjacent teeth and second preset parameters of distal adjacent teeth of the one or more teeth not grown to the preset eruption parameters.

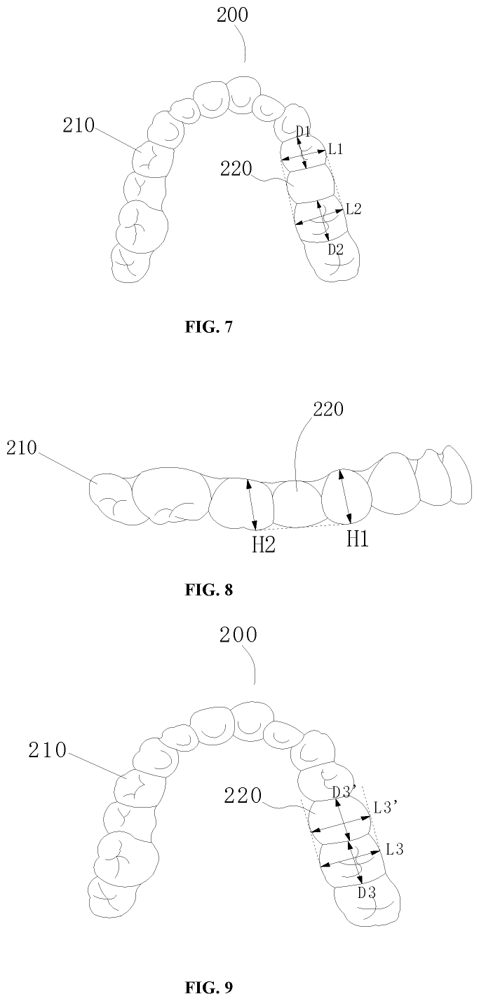

In some specific embodiments, the first preset parameters include the following parameters of the mesial adjacent teeth: a maximum dimension L₁ in a buccolingual diameter direction, a maximum dimension D₁ in a mesial-distal direction and a maximum dimension H₁ of a height of the tooth in a long axis direction. The second preset parameters include the following parameters of the distal adjacent teeth: a maximum dimension L₂ in the buccolingual diameter direction, a maximum dimension D₂ in the mesial-distal direction and a maximum dimension H₂ of the height of the tooth in the long axis direction. In one embodiment, as shown in FIG. 7 and FIG. 8, the eruption portion 220 of the shell-like dental appliance 200 is provided to encase eruption of a second premolar. At this time, the first preset parameters are a maximum dimension in the buccolingual diameter direction, a maximum dimension in the mesial-distal direction, and a maximum dimension of the height of the tooth in the long axis direction of a first premolar; the second preset parameters are a maximum dimension in the buccolingual diameter direction, a maximum dimension in the mesial-distal direction, and a maximum dimension of the height of the tooth in the long axis direction of the first premolar. The determination of the constant or substantially constant column structure based on the first preset parameters and the second preset parameters is performed by determining the dimension of the constant or substantially constant column structure in the buccolingual diameter direction by means of arithmetic averaging, weighted averaging, etc. between L₁ and L₂; determining the dimension of the constant or substantially constant column structure in the mesial-distal direction by means of arithmetic averaging, weighted averaging, etc. between D₁ and D₂; determining the dimensions of the constant or essentially constant column structure in the long axis of the teeth by means of arithmetic averaging, weighted averaging, etc. between H₁ and H₂. As a result, the eruption portion 220 may fit the size and shape of the one or more teeth not grown to the preset eruption parameters throughout the orthodontic plan to the maximum extent.

In some embodiments, the constant or substantially constant column structure includes a labial/buccal surface, a lingual surface, and an occlusal surface. The labial/buccal surface is a flat surface or a curved surface with a smooth transition to a labial/buccal surface of the mesial adjacent teeth and the distal adjacent teeth, the lingual surface is a flat surface or a curved surface with a smooth transition to a lingual surface of the mesial adjacent teeth and the distal adjacent teeth, and the occlusal surface is a flat surface or a curved surface with a smooth transition to an occlusal surface of the mesial adjacent teeth and the distal adjacent teeth. This setup structure allows the shell-like dental appliance 200 to have a smoother shell-like structure that wraps around the teeth and less foreign body sensation in the mouth when worn by the patient. It should be noted that the constant or substantially constant column structure means that the body shape, size, position, and orientation of the eruption portion 220 are consistent. Since the teeth may move as the orthodontic treatment proceeds during the orthodontic process of wearing a series of shell-like dental appliances, the eruption portion 120 may have a partial spatial adaptation for a smooth transition connection with a cavity of the adjacent teeth. In some embodiments, the constant or substantially constant column structure may only include the labial/buccal and lingual surfaces, but not include the occlusal surface.

In some embodiments, the constant or substantially constant column structure is further arranged based on the size, position, shape, and orientation of the teeth on the opposite jaw corresponding to the one or more teeth not grown to the preset eruption parameters so that the constant or substantially constant column structure does not interfere with the maxillomandibular occlusal relationship setting. The occlusal surface of the eruption portion 220 of this embodiment is designed as a flat, curved or a structure with protrusions and recesses to be matched with the opposing teeth. The occlusal surface of the eruption portion 220 is designed according to the opposing teeth so that the eruption portion 220 is cuspid-fossa matching with the opposing teeth, or so that the occlusal surface of this eruption portion 220 is a structure with protrusions and recesses to be matched with the occlusal surface of the opposing teeth.

In some embodiments, the constant or substantially constant column structure is arranged based on third preset parameters of distal adjacent teeth and mesial adjacent teeth of the one or more teeth not grown to the preset eruption parameters.

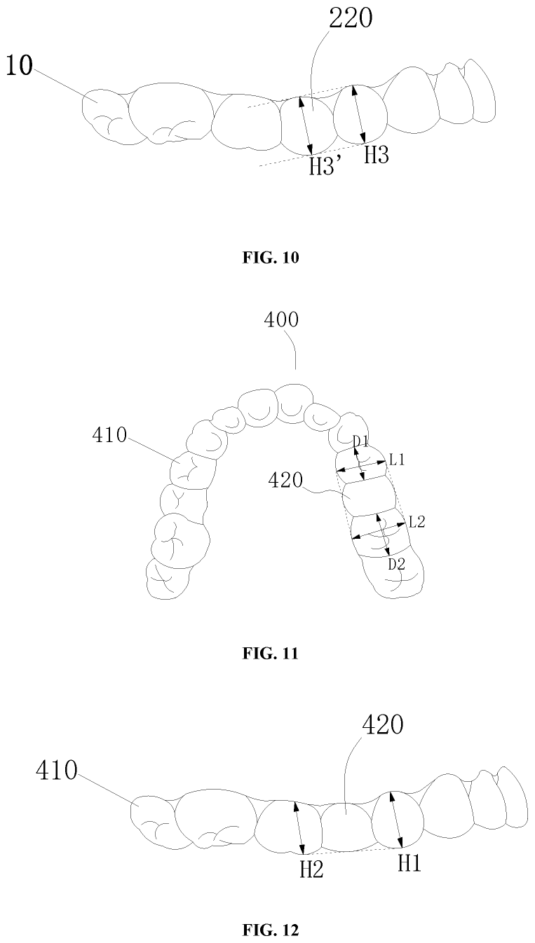

In some specific embodiments, the third preset parameters include: a maximum dimension in the buccolingual diameter direction, a maximum dimension in the mesial-distal direction of the distal adjacent teeth, and a maximum dimension of a height of a tooth in a long axis direction of the mesial adjacent teeth. In some specific embodiments, as shown in FIG. 9 and FIG. 10, the eruption portion 220 on the shell-like dental appliance is provided to encase the eruption of the second premolar. At this time, the third preset parameters are a maximum dimension L₃ in the buccolingual diameter direction, a maximum dimension D₃ in the medial-distal direction, and a maximum dimension H₃ of the height in the long-axis direction of the mesial adjacent teeth of the first molar, which may be used to determine a maximum dimension L₃′ in the buccolingual diameter direction, a maximum dimension D₃′ in the mesial-distal direction and a maximum dimension H₃′ in the height of the long-axis direction of the mesial adjacent teeth of the eruption portion 220 of the second premolar. Thus, the column structure designed on the basis of the maximum dimension in the buccolingual diameter direction of the distal adjacent teeth, the maximum dimension in the medial-distal direction and the maximum dimension of the height in the long-axis direction of the medial adjacent teeth is sufficient to accommodate the one or more teeth not grown to the preset eruption parameters, and such that the dimension of the column structure designed is slightly larger than that of the one or more teeth not grown to the preset eruption parameters, so as to ensure that a gap may be set between the column structure and the erupting tooth.

In some embodiments, the constant or substantially constant column structure includes a labial/buccal surface, a lingual surface, and an occlusal surface. The labial/buccal surface is a flat surface or a curved surface with a smooth transition to a labial/buccal surface of the mesial adjacent teeth and the distal adjacent teeth, the lingual surface is a flat surface or a curved surface with a smooth transition to a lingual surface of the mesial adjacent teeth and the distal adjacent teeth, and the occlusal surface is a flat surface or a curved surface with a smooth transition to an occlusal side of the mesial adjacent teeth and the distal adjacent teeth. This setup structure allows the shell-like dental appliance 200 to have a smoother shell-like structure that wraps around the teeth and less foreign body sensation in the mouth when worn by the patient.

In some embodiments, the constant or substantially constant column structure is further arranged based on the size, position, shape, and orientation of the teeth on the opposite jaw corresponding to the one or more teeth not grown to the preset eruption parameters so that the constant or substantially constant column structure does not interfere with the maxillomandibular occlusal relationship setting. The occlusal surface of the eruption portion 220 of this embodiment is designed as a flat, curved or a structure with protrusions and recesses to be matched with the opposing teeth. The occlusal surface of the eruption portion 220 may be designed according to the opposing teeth so that the eruption portion 220 is cuspid-fossa matching with the opposing teeth, or so that the occlusal surface of this eruption portion 220 is a structure with protrusions and recesses to be matched with the occlusal surface of the opposing teeth.

In some embodiments, the constant or substantially constant column structure is a cylindrical structure, an elliptical cylinder, or a multi-pronged column structure with not less than four lateral ribs, which may be set according to a number and type of erupting teeth, or may be adaptively selected according to an eruptive gap existing between adjacent teeth.

In some embodiments, a geometric structure on the shell-like dental appliance 200 other than the eruption portion 220 causes teeth other than unerupted teeth to be gradually adjusted from the initial position to the target orthodontic position. That is, the eruption portion 220 in this embodiment only reserves space for the growth of the one or more teeth not grown to the preset eruption parameters, so that the shell-like dental appliance 200 does not interfere with the natural growth of the erupting teeth, but the eruption portion 220 in this embodiment does not have a corrective effect on abnormally growing erupting teeth. That is, if the one or more teeth not grown to the preset eruption parameters are abnormally growing teeth, the eruption portion 220 of the present embodiment is also set according to the erupting teeth without corrective intervention. Instead, the geometric structure on the shell-like dental appliance 100 other than the eruption portion 120 causes the teeth other than the unerupted teeth to be gradually adjusted from the initial position to the target orthodontic position. That is, the geometric structure other than the eruption portion 120 has the corrective effect on the remaining teeth other than the erupted teeth, allowing the teeth to be aligned without interfering with the eruption of the teeth.

### Third Embodiment

This embodiment also provides a method for preparing an orthodontic system in which a shell-like dental appliance in an orthodontic system obtained according to anyone of the design methods described in the second embodiment is prepared by using a thermo-compression film molding or an additive preparing process to obtain a series of the shell-like dental appliances.

For example, when the thermos-compression film molding process is adopted, the specific preparing method includes: a 3D printing is carried out based on a digital dental model and a series of intermediate digital dental models to produce a solid dental model, after which the shell-like dental appliance containing a tooth shape is obtained by thermocompression film molding on the solid dental model, after which the shell-like dental appliance capable of accommodating teeth is subsequently obtained by cutting along or adjacent to a gingival line.

For example, when the additive preparing process is adopted, the specific preparing process is to print a digital model of the designed shell-like dental appliance using the 3D printing method.

### Fourth Embodiment

This embodiment provides a method for predicting an eruption cavity of a shell-like dental appliance, in which at least one shell-like dental appliance 400 is designed to gradually adjust teeth from an initial position to a target orthodontic position while allowing the teeth to erupt according to an orthodontic plan. Herein, the shell-like dental appliance 400 includes a dental appliance body 410 with a geometric structure accommodating multiple maxillary teeth or multiple mandibular teeth and at least one eruption cavity 420 accommodating one or more teeth not grown to preset eruption parameters. The eruption cavity 420 is predicted based on both first preset parameters of mesial adjacent teeth and second preset parameters of distal adjacent teeth of the one or more teeth not grown to the preset eruption parameters, such that a gap is set between an inner surface of the eruption cavity 420 and an outer surface of the teeth not grown to the preset eruption parameters. The preset eruption parameters are designed to include parameters of a tooth or teeth formed after full eruption of one or more ungrown or incompletely grown teeth.

In some embodiments, the preset eruption parameters include a size, a position, a shape, and an orientation of the tooth or teeth formed after the full eruption of the one or more ungrown or incompletely grown teeth. Herein, the size, position, shape, and orientation may be a size, position, shape, and orientation of the incompletely grown teeth obtained based on CBCT of a patient, or a size, position, shape, and orientation of the ungrown or incompletely grown teeth obtained based on one or more denture libraries, or a size, position, shape, and orientation of the ungrown or incompletely grown tooth based on statistics under big data.

In some embodiments, the first preset parameters include the following parameters of the mesial adjacent teeth: a maximum dimension in a buccolingual diameter direction, a maximum dimension in a mesial-distal direction, and a maximum dimension of a height of a tooth in a long axis direction. The second preset parameters include the following parameters of the distal adjacent teeth: a maximum dimension in the buccolingual diameter direction, a maximum dimension in the mesial-distal direction, and a maximum dimension of the height of the tooth in the long axis direction.

In some specific embodiments, the first preset parameters include the following parameters of the mesial adjacent teeth: a maximum dimension L₁ in the buccolingual diameter direction, a maximum dimension D₁ in the mesial-distal direction and a maximum dimension H₁ of the height of the tooth in the long axis direction. The second preset parameters include the following parameters of the distal adjacent teeth: a maximum dimension L₂ in the buccolingual diameter direction, a maximum dimension D₂ in the mesial-distal direction and a maximum dimension H₂ of the height of the tooth in the long axis direction. In one embodiment, as shown in FIG. 11 and FIG. 12, the eruption cavity 420 on the shell-like dental appliance 400 is provided to encase eruption of a second premolar. At this time, the first preset parameters are a maximum dimension in the buccolingual diameter direction, a maximum dimension in the mesial-distal direction, and a maximum dimension in the height of the tooth in the long axis direction of a first premolar; the second preset parameters are a maximum dimension in the buccolingual diameter direction, a maximum dimension in the mesial-distal direction, and a maximum dimension of the height of the tooth in the long axis direction of a first molar. And the eruption cavity 420 is determined based on the first preset parameters and the second preset parameters is performed by determining the dimension of the eruption cavity 420 in the buccolingual diameter direction by means of arithmetic averaging, weighted averaging, etc. between L1 and L2; determining the dimension of the eruption cavity 420 in the mesial-distal direction by means of arithmetic averaging, weighted averaging, etc. between D1 and D2; determining the dimension of the eruption cavity 420 in the long axis direction of the teeth by means of arithmetic averaging, weighted averaging, etc. between H1 and H2. As a result, the eruption cavity 420 may fit the size and shape of the one or more teeth not grown to the preset eruption parameters throughout the orthodontic plan to the maximum extent.

In some embodiments, the eruption cavity 420 includes a labial/buccal surface, a lingual surface, and an occlusal surface. The labial/buccal surface is a flat surface or a curved surface with a smooth transition to a labial/buccal surface of the mesial adjacent teeth and the distal adjacent teeth, the lingual surface is a flat surface or a curved surface with a smooth transition to a lingual surface of the mesial adjacent teeth and the distal adjacent teeth, and the occlusal surface is a flat surface or a curved surface with a smooth transition to an occlusal surface of the mesial adjacent teeth and the distal adjacent teeth. This setup structure allows the shell-like dental appliance 400 to have a smoother shell-like structure that wraps around the teeth and less foreign body sensation in the mouth when worn by a patient.

In some embodiments, the eruption cavity 420 is also arranged based on a size, a position, a shape, and an orientation of teeth on an opposite jaw corresponding to the one or more teeth not grown to the preset eruption parameters, so that the structure of the eruption cavity 420 does not interfere with maxillomandibular occlusal relationship setting. The occlusal surface of the eruption cavity 420 of this embodiment is designed as a flat, curved or a structure with protrusions and recesses to be matched with opposing teeth. The occlusal surface of the eruption cavity 420 may be designed according to the opposing teeth so that the eruption cavity 420 is cuspid-fossa matching with the opposing teeth, or so that the occlusal surface of this eruption cavity 420 is a structure with protrusions and recesses to be matched with the occlusal surface of the opposing teeth.

### Fifth Embodiment

This embodiment provides a method for predicting an eruption cavity of a shell-like dental appliance, in which at least one shell-like dental appliance 500 that gradually adjusts teeth from an initial position to a target orthodontic position while allowing the teeth to erupt according to an orthodontic plan is designed. Herein, one shell-like dental appliance 500 includes a dental appliance body 510 with a geometric structure accommodating multiple maxillary teeth or multiple mandibular teeth and at least one eruption cavity 520 accommodating one or more teeth not grown to the preset eruption parameters. The eruption cavity 520 is predicted based on third preset parameters of distal adjacent teeth and mesial adjacent teeth of the one or more teeth not grown to the preset eruption parameters, such that a gap is set between an inner surface of the eruption cavity 520 and an outer surface of the teeth not grown to the preset eruption parameters. The preset eruption parameters are designed to include parameters of a tooth or teeth formed after full eruption of one or more ungrown or incompletely grown teeth.

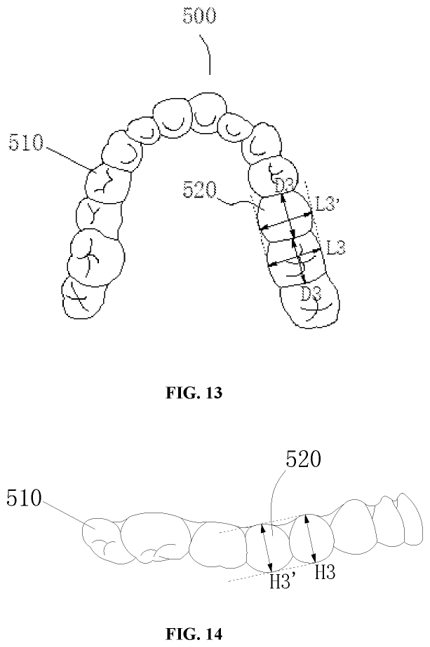

In some embodiments, the third preset parameters include: a maximum dimension in a buccolingual diameter direction, a maximum dimension in a mesial-distal direction of the distal adjacent teeth, and a maximum dimension of a height of a tooth in a long axis direction of the mesial adjacent teeth. In some specific embodiments, as shown in FIG. 13 and FIG. 14, the eruption cavity 520 on the shell-like dental appliance 500 is provided to encase eruption of a second premolar. At this time, the third preset parameters include: a maximum dimension L₃ in a buccolingual diameter direction, a maximum dimension D₃ in a mesial-distal direction, and a maximum dimension H₃ of a height in a long axis direction of the mesial adjacent teeth of a first molar, which is used to determine a maximum dimension L₃′ in the buccolingual diameter direction, a maximum dimension D₃′ in the mesial-distal direction, and a maximum dimension H₃′ in the height of the long axis direction of the mesial adjacent teeth of the eruption cavity 520 of the second premolar. The eruption cavity 520 designed based on the maximum dimension in the buccolingual diameter direction of the distal adjacent teeth, the maximum dimension in the mesial-distal direction and the maximum dimension of the height in the long axis direction of the mesial adjacent teeth is sufficient to accommodate the one or more teeth not grown to the preset eruption parameters and such that the dimension of the eruption cavity 520 designed is slightly larger than that of the one or more teeth not grown to the preset eruption parameters, so as to ensure that a gap is set between the eruption cavity 520 and the erupting teeth.

In some embodiments, the eruption cavity 520 includes a labial/buccal surface, a lingual surface, and an occlusal surface. Herein, the labial/buccal surface is a flat surface or a curved surface with a smooth transition to a labial/buccal surface of the mesial adjacent teeth and the distal adjacent teeth; the lingual surface is a flat surface or a curved surface with a smooth transition to a lingual surface of the mesial adjacent teeth and the distal adjacent teeth, and the occlusal surface is a flat surface or a curved surface with a smooth transition to an occlusal surface of the mesial adjacent teeth and the distal adjacent teeth. This setup structure allows the shell-like dental appliance 500 to have a smoother shell-like structure that wraps around the teeth and less foreign body sensation in the mouth when worn by the patient.

In some embodiments, the eruption cavity 520 is further arranged based on a size, a position, a shape, and an orientation of teeth on an opposite jaw corresponding to the one or more teeth not grown to the preset eruption parameters so that the structure of the eruption cavity 520 does not interfere with maxillomandibular occlusal relationship setting. The occlusal surface of the eruption cavity 520 of this embodiment is designed as a flat, curved or a structure with protrusions and recesses to be matched with opposing teeth. The occlusal surface of the eruption cavity 520 is designed based on the opposing teeth so that the eruption cavity 520 is cusp-fossa matching with the opposing teeth, or so that the occlusal surface of the eruption cavity 520 is a structure with protrusions and recesses to be matched with the occlusal surface of the opposing teeth.

In some embodiments, the eruption cavity 520 is a cylindrical structure, an elliptical cylinder, or a multi-prismatic structure with not less than four lateral ribs, which may be set according to a number and type of missing teeth, or may be adaptively selected according to an eruptive gap existing between adjacent teeth.

The disclosed embodiments above are only preferred embodiments of the present disclosure, and the preferred embodiments do not elaborate on all the details. It should be understood that these embodiments are used only to illustrate the present disclosure and not to limit the scope of protection of the present disclosure, which is limited only by the claims and their full scope and equivalents.

These embodiments are selected and specifically described in this specification to better explain the principles and practical applications of the present disclosure, so that those skilled in the art can make good use of the present disclosure. The technical features in the above different embodiments can be arbitrarily combined on the premise without conflicting with each other, and the improvements and adjustments made by those skilled in the art in accordance with the present disclosure in practical applications still fall within the scope of protection of the present disclosure.

What is claimed is:

1. An orthodontic system comprising at least one shell-like dental appliance configured to gradually adjust each of teeth other than unerupted teeth from an initial position to a target orthodontic position according to an orthodontic plan while allowing the teeth to erupt naturally;
wherein each of the at least one shell-like dental appliance comprises a dental appliance body comprising a geometric structure for accommodating a plurality of maxillary teeth or a plurality of mandibular teeth, and the dental appliance body is further provided with at least one eruption portion for accommodating one or more teeth not grown to preset eruption parameters; as the orthodontic plan proceeds, the eruption portion on each of the at least one shell-like dental appliances has a constant or substantially constant column structure, and there is a gap between an inner surface of each of the at least one eruption portion and an outer surface of each of the one or more teeth not grown to preset eruption parameters; wherein the preset eruption parameters comprise parameters of a tooth or teeth formed after full eruption of one or more ungrown or incompletely grown teeth.

2. The orthodontic system according to claim 1, wherein the preset eruption parameters comprise a size, a position, a shape and an orientation of the tooth or teeth formed after the full eruption of the one or more ungrown or incompletely grown teeth.

3. The orthodontic system according to claim 1, wherein the constant or substantially constant column structure is arranged based on a size, a position, a shape and an orientation of a tooth or teeth formed after full eruption of the one or more teeth not grown to the preset eruption parameters.

4. The orthodontic system according to claim 3, wherein the column structure has a size 1.02 to 1.05 times of a size of the tooth or teeth formed after the full eruption of the one or more teeth not grown to the preset eruption parameters; the column structure is oriented at an angle of 0 to 5 degrees to a long axis of the tooth or teeth formed after the full eruption of the one or more teeth not grown to the preset eruption parameters; the column structure is positioned at an offset of 0 to 1 mm from a coordinate value in a spatial three-dimensional coordinate system of each of vertices that constitute a position of the tooth or teeth formed after the full eruption of the one or more teeth not grown to the preset eruption parameters; the column structure is shaped at an offset of 0 to 1 mm from a coordinate value in the spatial three-dimensional coordinate system of each of vertices that constitute a shape of the tooth or teeth formed after the full eruption of the one or more teeth not grown to the preset eruption parameters.

5. The orthodontic system according to claim 3, wherein the constant or substantially constant column structure is further arranged based on a size, a position, a shape and an orientation of a tooth or teeth on an opposite jaw corresponding to the one or more teeth not grown to the preset eruption parameters, so that the constant or substantially constant column structure does not interfere with a maxillomandibular occlusal relationship.

6. The orthodontic system according to claim 1, wherein the constant or substantially constant column structure is arranged based on both first preset parameters of mesial adjacent teeth and second preset parameters of distal adjacent teeth of the one or more teeth not grown to the preset eruption parameters.

7. The orthodontic system according to claim 6, wherein the first preset parameters comprise following parameters of the mesial adjacent teeth: a maximum dimension in a buccolingual diameter direction, a maximum dimension in a mesial-distal direction, and a maximum dimension of a height of a tooth in a long axis direction; the second preset parameters comprise the following parameters of the distal adjacent teeth: a maximum dimension in the buccolingual diameter direction, a maximum dimension in the mesial-distal direction, and a maximum dimension of the height of the tooth in the long axis direction.

8. The orthodontic system according to claim 7, wherein the constant or substantially constant column structure comprises a labial/buccal surface, a lingual surface and an occlusal surface, wherein the labial/buccal surface is a flat surface or a curved surface with a smooth transition to a labial/buccal surface of the mesial adjacent teeth and the distal adjacent teeth, the lingual surface is a flat surface or a curved surface with a smooth transition to a lingual surface of the mesial adjacent teeth and the distal adjacent teeth, and the occlusal surface is a flat surface or a curved surface with a smooth transition to an occlusal surface of the mesial adjacent teeth and the distal adjacent teeth.

9. The orthodontic system according to claim 6, wherein the constant or substantially constant column structure is further arranged based on a size, a position, a shape, and an orientation of teeth on an opposite jaw corresponding to the one or more teeth not grown to the preset eruption parameters, so that the constant or substantially constant column structure does not interfere with a maxillomandibular occlusal relationship.

10. The orthodontic system according to claim 1, wherein the constant or substantially constant column structure is arranged based on third preset parameters of distal adjacent teeth and mesial adjacent teeth of the one or more teeth not grown to the preset eruption parameters.

11. The orthodontic system according to claim 10, wherein the third preset parameters comprise: a maximum dimension in a buccolingual diameter direction, a maximum dimension in a medial-distal direction of the distal adjacent teeth, and a maximum dimension of a height of a tooth in a long axis direction of the mesial adjacent teeth.

12. The orthodontic system according to claim 10, wherein the constant or substantially constant column structure comprises a labial/buccal surface, a lingual surface and an occlusal surface, wherein the labial/buccal surface is a flat surface or a curved surface with a smooth transition to a labial/buccal surface of the mesial adjacent teeth and the distal adjacent teeth and distal adjacent teeth, the lingual surface is a flat surface or a curved surface with a smooth transition to a lingual surface of the mesial adjacent teeth and the distal adjacent teeth, and the occlusal surface is a flat surface or a curved surface with a smooth transition to an occlusal surface of the mesial adjacent teeth and the distal adjacent teeth.

13. The orthodontic system according to claim 10, wherein the constant or substantially constant column structure is further arranged based on a size, a position, a shape, and an orientation of teeth on an opposite jaw corresponding to the one or more teeth not grown to the preset eruption parameters, so that the constant or substantially constant column structure does not interfere with a maxillomandibular occlusal relationship.

14. The orthodontic system according to claim 1, wherein the constant or substantially constant column structure is a cylindrical structure, an elliptical cylindrical structure, or a multi-prismatic column structure with not less than four lateral ribs.

15. An orthodontic system according to claim 1, wherein the geometric structure on the at least one shell-like dental appliance other than the eruption portion is configured to gradually adjust each of the teeth other than the unerupted teeth from the initial position to the target orthodontic position.

16. A design method of an orthodontic system, wherein the design method comprises:
S1. obtaining a digital dental model: obtaining the digital dental model, wherein the digital dental model comprises a digital teeth model and a digital gingival model;
S2. segmenting and identifying the digital dental model: segmenting the digital dental model into the digital gingival model and a plurality of digital tooth crown models in a one-to-one correspondence to the teeth; identifying and marking data indicating teeth which do not erupt or do not fully erupt;
S3. virtually designing an orthodontic plan: designing the plurality of digital tooth crown model in a one-to-one correspondence to the teeth virtually so that each of the plurality of digital tooth crown model in a one-to-one correspondence to the teeth gradually changes from an initial position to a target orthodontic position to obtain a series of intermediate digital dental models;
S4. designing the orthodontic system:
designing at least one shell-like dental appliance configured to gradually adjust each of the teeth from the initial position to the target orthodontic position according to the orthodontic plan while allowing the teeth to erupt naturally, wherein each of the at least one shell-like dental appliance comprises a dental appliance body comprising a geometric structure for accommodating a plurality of maxillary teeth or a plurality of mandibular teeth, and the dental appliance body is further provided with at least one eruption portion for accommodating one or more teeth not grown to preset eruption parameters;
as the orthodontic plan proceeds, the eruption portion on each of the at least one shell-like dental appliances has a constant or substantially constant column structure, and there is a gap between an inner surface of each of the at least one eruption portion and an outer surface of each of the one or more teeth not grown to preset eruption parameters; wherein the preset eruption parameters comprise parameters of a tooth or teeth formed after full eruption of one or more ungrown or incompletely grown teeth.

17. A design method of an orthodontic system according to claim 16, wherein the geometric structure on the at least one shell-like dental appliance other than the eruption portion is configured to gradually adjust each of the teeth other than the unerupted teeth from the initial position to the target orthodontic position.

18. A method for preparing an orthodontic system, wherein the at least one shell-like dental appliance in the orthodontic system obtained according to the design method according to claim 16 is prepared by using a thermo-compression molding or an additive preparing process to obtain a series of the shell-like dental appliances.

19. A method for preparing an orthodontic system, wherein the at least one shell-like dental appliance in the orthodontic system obtained according to the design method according to claim 17 is prepared by using a thermo-compression molding or an additive preparing process to obtain a series of the shell-like dental appliances.
